(19) United States
(12) Patent Application Publication
(10) Pub. No.: US 2003/0190339 A1
(43) Pub. Date: Oct. 9, 2003

(54) NOVEL SKIN RESURFACING RECOVERY SYSTEM

(76) Inventors: GREGORY SKOVER, PRINCETON, NJ (US); DANIEL FORMOSA, MONTVALE, NJ (US); HENRI BRISEBOIS, QUEBEC (CA); ZULFIKAR MURJI, VERDUN (CA); ISABELLE LEGALL, MONTREAL (CA)

Correspondence Address: AUDLEY A CIAMPORCERO JR, JOHNSON AND JOHNSON, ONE JOHNSON AND JOHNSON PLAZA, NEW BRUNSWICK, NJ, 089337003

(21) Appl. No.: 09/170,455

(22) Filed: Oct. 13, 1998

Related U.S. Application Data
(60) Provisional application No. 60/061,762, filed on Oct. 14, 1997.

(51) Int. Cl. A61F 13/00; A61F 15/00; A01N 25/34

(52) U.S. Cl. 424/402; 424/445; 602/45; 602/47

(57) ABSTRACT

This invention relates to methods of promoting the healing of skin wounds, products for promoting the healing of skin wounds and processes for making such products. More particularly, this invention relates to layered absorbent dressings which absorb wound exudate in conjunction with additional products for assisting the healing of wounds at each stage of wound healing, as well as methods of using these dressings and methods of making these dressings.

### 1. FIELD OF THE INVENTION

This invention relates to apparatus, compositions and methods for healing skin which has been treated with resurfacing techniques. More particularly, it relates to dressings, compositions and apparatus which can be applied to skin after resurfacing surgery and which hasten the healing process.

### 2. BACKGROUND OF THE INVENTION

Cosmetic dermatology is currently experiencing explosive growth due to an increasing patient population and expansion of options in the procedures and practices that deliver more youthful-looking skin. However, no product or regimen exists today that optimizes the skin's healing environment following these advanced treatment procedures.

Returning the skin to a functional barrier following laser resurfacing, deep chemical peeling and dermabrasion proceeds through three distinct phases: an early inflammatory phase, followed by a proliferative phase and continuing through a remodeling phase. Each step progressively evolves into the subsequent step, ultimately leading to a daily maintenance regimen.

### SUMMARY OF THE INVENTION

The compositions, apparatus and processes of this invention provide a wound management system that meets the needs of both clinicians and patients following cosmetic resurfacing procedures. This system is based on the mechanisms of wound healing. A primary and/or secondary dressing is applied to the skin which absorbs fluid and wound exudate upon completing skin treatment, during the early inflammatory phase. During the next healing phase, the proliferative phase, another maintenance dressing is applied. During the remodeling phase, a barrier material is applied to preserve the barrier function of the healing skin. Benefits to clinicians are: optimization of wound repair, minimization of patient confusion/anxiety, maximization of patient satisfaction. Benefits to patients are: increased comfort and control over their post-treatment management, development of their maintenance skin care program. The compositions, apparatus and processes of this invention may be utilized in wound management as well as in any situation in which it is desirable to cover an area of skin, particularly the facial area. Such situations may include; facial skin treatments such as laser resurfacing, facial peels and moisturization of facial tissue.

The compositions, apparatus and processes of this invention provide a progressive healing system that provides the optimal environment enabling healing to proceed at a rapid rate and meets the specific needs of skin through each stage of healing. The products of this invention are easy to use in the patient's home and are very comfortable. In accordance with the compositions, apparatus and processes of this invention, the skin is returned to a normal, healthy state within about six days after the resurfacing treatment is performed.

Generally, there are three major phases in returning skin to a functional barrier following skin resurfacing procedures: (1) early inflammatory phase, which produces a large amount of wound fluid or exudate that must be managed effectively to maintain the optimal would environment while providing patient comfort; (2) the proliferative phase, in which the epidermis is regenerated and the wound must be maintained at the optimal temperature and humidity to facilitate healing and minimize patient discomfort; and (3) reestablishing the barrier properties of the skin. The processes and products of this invention address all of the needs of the patient during each phase of the healing process.

### DETAILED DESCRIPTION OF PREFERRED EMBODIMENTS

In accordance with this invention, a method, compositions and methods of manufacture are is provided for promoting wound healing and skin treatment. The method of treating skin comprises the following steps:

(A) applying one of the following products to the skin:

(i) a system comprising at least the following layers:

(a) a primary absorptive skin-facing layer for the purpose of absorbing wound fluid or exudate during the early inflammatory phase of healing to maintain the optimal wound environment and to provide patient comfort; and

(b) a secondary absorptive cover layer in association with said primary absorptive skin-facing layer which is breathable and attaches to the face and the primary absorptive layer; said primary and secondary absorptive layers being applied for approximately 24 to 36 hours after resurfacing surgery; or

(ii) an absorbent layer having the characteristics of absorbing fluid from the wound without adhering to the wound and which is sufficiently breathable to permit vapor to escape from the wound or skin site covered;

(B) removing said covering layers and applying a maintenance layer which regulates the temperature and humidity of the wound environment during the proliferative phase of wound healing for approximately 24 to 36 hours after removing the primary and secondary absorptive layers; and

(C) removing said maintenance layer and applying a barrier material which minimizes trans-epidermal water loss and enhances intercellular lipid fluidity of the skin during the phase in which the barrier properties of the skin are reestablished.

One of the most important aspects of the methods and apparatus of this invention is the first dressing which may be placed upon the desired area. This dressing should be highly absorbent, yet breathable, permitting wound exudate and/or moisture to be drawn from the skin yet regulating the temperature and humidity of the skin. One of the preferred embodiments of the dressings of this invention can be composed of two or more layers, a primary and a secondary absorptive layer. Alternately, it may be a laminate or combination material incorporating both a primary and secondary dressing.

Preferably, when the dressing is composed of at least two discrete layers, the primary absorptive layer is composed of a material which is highly absorbent and pliable so as to conform well to the contours of the face. More preferably, the primary absorptive layer is composed of a lyophilized collagen-alginate wound dressing. Most preferably, the primary absorptive layer is composed of Fibracol*, a lyophilized collagen-alginate wound dressing currently available from Johnson & Johnson Medical, Inc. of Arlington, Tex. Fibracol* is currently marketed for use in treating partial thickness wounds. We have found it to be extremely useful in managing the early inflammatory stage of healing of resurfaced skin.

The soft, light pliable matrix of lyophilized collagen and alginate provide immediate exudate absorption upon application. As the Fibracol primary dressing absorbs exudate, it conforms to the contours of the face, creating an intimate protective layer over the denuded dermis. The presence of collagen acts as a hemostat, enhancing coagulation and the release of repair-enhancing peptides from platelets. As Fibracol absorbs exudate, it becomes a gel, confining the natural growth factors to the surface of the injured skin, facilitating skin regeneration. The absorptive layer composed of Fibracol* absorbs up to 70% times its weight in exudate. The primary absorptive layer may also be composed of a hydrogel layer, ointment or any other wound dressing or skin treatment composition known to those of ordinary skill in the art.

Preferably, the primary absorptive layer is associated with a secondary absorptive layer which acts as a cover to the primary absorptive layer. The breathable absorbent cover is preferably a soft, flexible cover that maximizes the benefits of the primary absorptive layer. It should be ergonomically designed to conform to the face and to attach to the primary absorptive layer.

The breathable, absorbent cover assists the primary absorptive layer in fluid management and creates an optimal wound environment that facilitates epidermal regeneration. At the end of the inflammatory phase, the dressing may be removed without pain or re-injury and the facial skin is ready to proceed to the regeneration phase of healing. The breathable secondary absorptive layer allows water vapor to pass through while preventing the leaking of moisture, thus substantially preventing the skin from drying while maintaining a healthy exchange of water vapor and air to the wound. The secondary absorptive layer also assists in the easy removal of the primary absorptive layer upon completion of the first phase of healing.

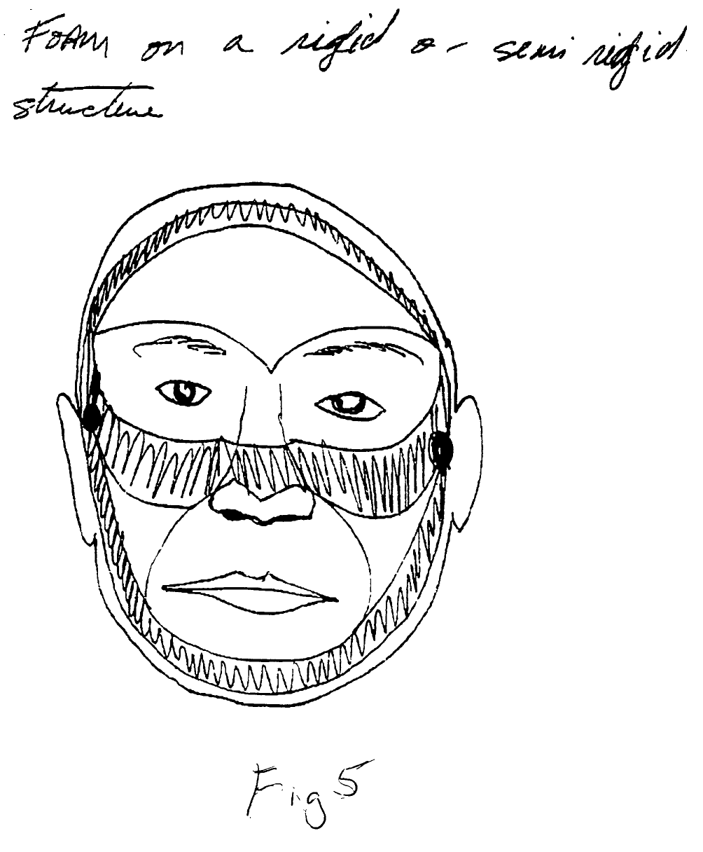

The secondary absorptive layer that can be used in conjunction with said primary absorptive layer or as a primary absorptive layer by itself is preferably a laminate composed of at least one absorbent material which may be a nonwoven fluff pulp or fibrous material and at least one thermoplastic or thermofusable material. The secondary absorptive layer should be breathable, and thus porous. Alternatively, the secondary absorptive layer may be composed of two or more materials which have been heat-sealed, embossed and perforated (hereinafter, “perf-embossing”) so as to provide pores and depressions in a composite layer. The absorbent material should contain fibers that can be fused by heat during the embossing process in the thermofusable absorbent layer. IT may be provided as a batt or as a nonwoven fabric material. The perf-embossing process creates “wells”, or depressions in the composite, as well as perforations, slits, openings, stretches or fractures on the side walls of the wells. The slits or openings allow liquid to flow easily into the absorbent structure, while the dosed bottom-of the wells impedes fluid strikeback. The slits or openings of the embossments of the products of this invention may be seen in FIGS. 5a, 5b and 5c.

The primary and secondary absorptive layers should be bendable and conformable so as to be able to conform with the three-dimensional topography of the face, for example, without causing additional trauma to the delicate tissue being healed.

Preferably, the absorbent core material contains thermofusible fibers that can bind when heated with films placed on a body-facing side of the core material and, optionally, on the outer-facing side of the core material. The fibers may be bicomponent fibers, for example, composed of a polyester core with a polyethylene sheath or a polypropylene core with a polyethylene sheath or like bonding fibers known to those of skill in the art so long as they can be heat-fused with other materials and maintain its structure. Such fibers are capable of being bonded to each other and to the film facings and maintain the integrity of the primary and secondary absorptive layers. Furthermore, thermofusible fibers permit holes to be made in the layers to provide openings which can be heat-sealed for the nose, mouth and eyes to create a mask for applying the dressing to the face. The openings in the composite dressing can be fused and sealed with heat and form barriers to the fluids being absorbed into the dressing, thus preventing them from flowing toward the eyes, nose and mouth, providing a comfortable dressing for the patient.

The absorbent core layer of the secondary absorptive layer of the products of this invention preferably contain fluff pulp and a bonding fiber. Preferably, the absorbent laminate layer should contain from about 5 to about 40% thermofusible bonding fiber, most preferably from about 20 to about 25%. The basis weight of the absorbent laminate layer should be from about 50 to about 200 grams per square meter, more preferably from about 100 to 150 and most preferably from about 110 to about 125. The density should be from about 0.03 to about 0.2 g/cc, more preferably from about 0.07 to about 0.10 and most preferably from about 0.07 to about 0.09 g/cc, preferably, Concert 120.899 available from the Concert Company of Thurso, Canada.

The body-facing side of the secondary absorbent composite layer should preferably be laminated on its body-facing side to an acquisition facing. This acquisition facing is preferably a relatively non-stretchable film which can be cleanly perforated. Preferably, the film should have perforations in a pattern containing a straight edge, although they can be in the shape of ellipses or arcs so long as they provide slits rather than macropores. The acquisition facing provides a means through which wound fluid and exudate may be acquired by the absorbent core layer and removed from the immediate vicinity of the skin. This aids and expedites the healing process without adhering to the healing skin and causing trauma upon removal, as would a fibrous absorbent batt or non-woven material. Preferably, a composite film containing polyethylene and ethylene vinyl acetate from about 0.5 to about 1.5 mil in thickness prior to embossing may be used. The film may have a thickness of about 3 to 4 mils, preferably 3.5 mils as supplied, as the thickness increases after embossment by the manufacturer. Preferably, the body-facing acquisition facing is a coextruded polymer film, most preferably polyethylene/ethylene vinyl acetate, such as XP-1167B commercially available from Edison Plastics of Edison, N.J. Breaking Elongation of the film in the machine direction=425+/−225 and cross direction 685+/−155 in accordance with ASTM D882. The Tensile strength@25% is: MD 640+/−152, CD 470+/−85. These numbers are for a 1 mil film.

Other materials that can be used on the absorbent composite are: Reticulon* perforated film of 1.0 oz/yd² from PGI and Enka nonwoven fabric containing thermofusible fibers and having a weight of from about 0.5 to about 2 oz/yd² commercially available from PGI of Dayton, N.J. The outward-facing side of the secondary absorbent composite layer should be associated with a barrier film layer. This layer is preferably laminated to the absorbent material. It should be porous, such that moisture can escape through it away from the skin. Preferably, the barrier film layer should be either monolithic (e.g. polyurethane or polyester or the like) or microporous, such that the layer is breathable. The absorbent composite layer may be laminated to the barrier film layer using heat, if both the film layer and components of the absorbent layer are thermofusible. Alternatively, they may be laminated with a hot melt adhesive spray.

More preferably, breathable microporous film should be used. The film should have a weight of from about 20 to about 40 gsm, more preferably from about 25 to about 30 gsm. The Moisture Vapor Transmission Rate (MVTR) should be in the range of from about 500 to about 7500 g/m²/day, more preferably from about 750 to about 4000 g/m²/day and most preferably from about 1000 to about 3000 g/m2/day as tested using test method ASTM E96-90 or E96-E of Clopay of Cincinnati, Ohio.

Polyurethane and other breathable films that have the appropriate MVTR may also be utilized in the composite of this invention. Most preferably, the film should be a polyethylene microporous film. The products of the invention as currently tested used a polyethylene microporous film known and available as Br-200 or Br-300 from Clopay of Cincinnati, Ohio.

More preferably, the composite layers of the secondary composite layer should be laminated using hot melt lamination adhesive, at a quantity of from about 0.5 mg/in² to about 3.5 mg/in2. Most preferably, it should be used in an amount of from about 1.75 to about 2.25 mg/in2. Any hot melt adhesive known to those of skill in the art should be effective to adhere the layers. The thickness of completed absorbent composite should be from about 0.075 in. to about 0.1 in. at 0.1 psi. The MVTR of the complete absorbent composite should be from about 500 to 3000 g/m²/day. More preferably, it should be from about 1000 to about 2000 g/m²/day using test method ASTM E96-90 or E96-E of Clopay of Cincinnati, Ohio.

The absorbent composite of this invention may be utilized either as a secondary dressing in conjunction with a primary dressing such as a hydrogel, ointment or other similar type of dressing material. Alternatively, it may be utilized itself as a primary dressing alone, as it incorporates the wound environment management characteristics and breathability characteristics required by the products of this invention. Furthermore, it can function as a cover to any solvent-based skin treatment, promoting the penetration of the treatment to achieve the desired effect. Skin treatments may include peels, moisturization using alpha hydroxyacids, botanical-type moisturizers and rejuvenators and the like for enhancement of penetration into the skin and protection of clothing and ambient environment from the skin treatment compositions. The composite laminate product of this invention may also be used in many types of absorbent products, including wound dressings, panty liners, sanitary napkins, incontinence products, diapers, and the like.

The absorbent composite of this invention may be obtained when two or more materials are fed through a pair of heated cylindrical embossing rolls having projections or knuckles (e.g., diamond or hexagonal) in intermeshing arrangements. The composite has at least one material that is thermoplastic and at least one layer is a nonwoven/absorbent material. The resulting composite is a highly flexible, soft and conformable, bulky three-dimensional laminate with raised and depressed areas on the surface. The raised and depressed areas effectively create wells on both surfaces of the composite. The perf-embossing process creates perforations, slits, openings, stretches or fractures on “side walls” of the wells. The slits or openings allow liquid to flow easily into the absorbent structure, while the “closed” bottom of the wall impedes fluid strikeback. Perf-embossing processes such as that described herein are also set forth in U.S. Pat. No. 5,242,435 (Murji and Brisebois) and U.S. Pat. No. 3,817,827 (Benz) which are hereby incorporated herein by reference.

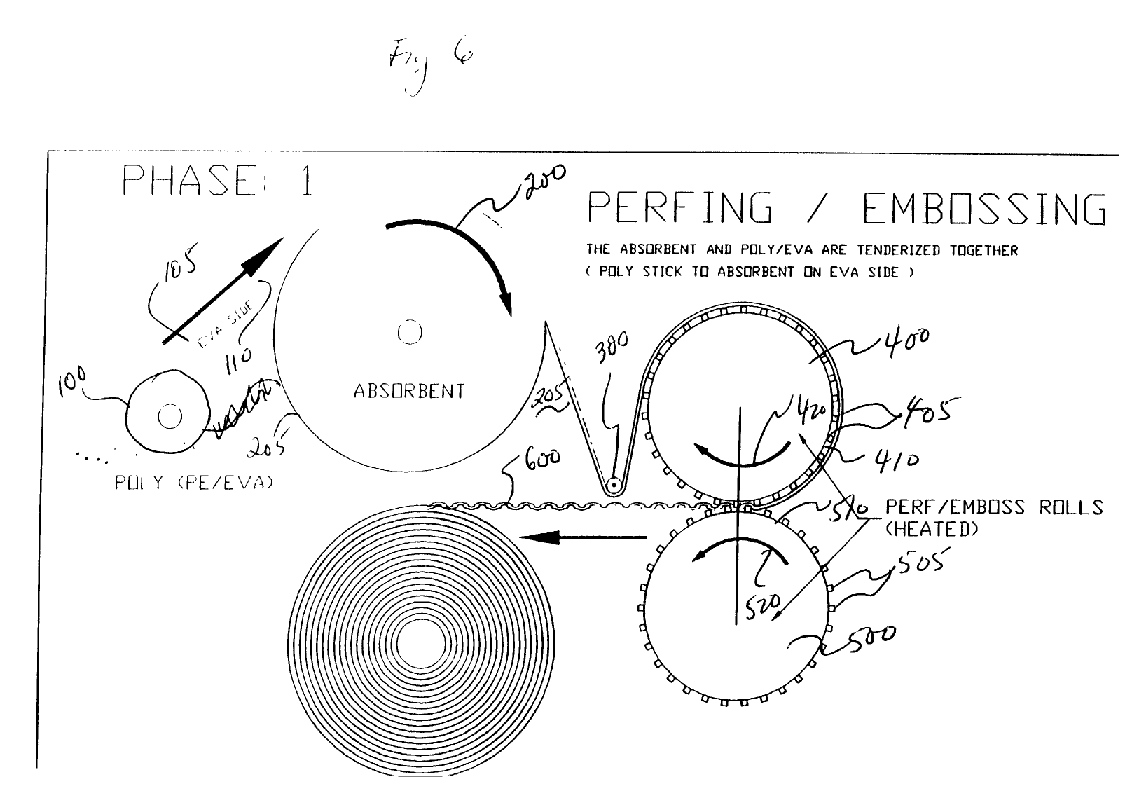

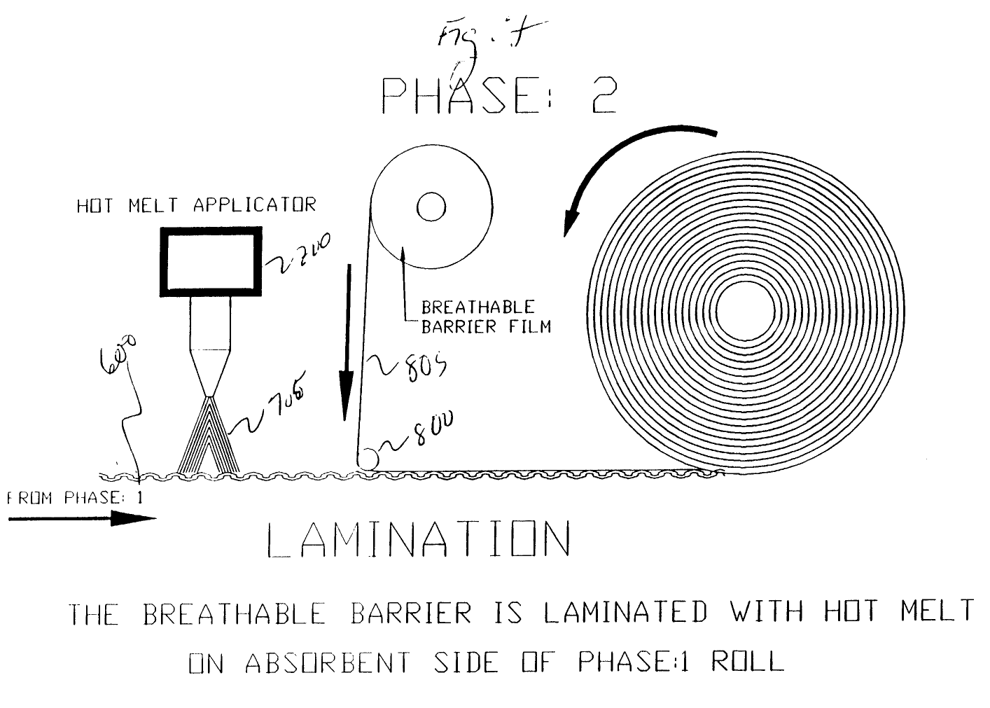

The perf embossing process for making the secondary absorbent layer of the products of this invention is performed as follows. Referring to FIG. 6, a roll 100 containing a polyethylene/ethylene vinyl acetate film 105 is unwound over a roll 200 containing an absorbent layer 205, such that the ethylene vinyl acetate side of the film 110 is contiguous with absorbent layer 205, as set forth in FIG. 6. The film 105 and absorbent layer 205 are wound under a weighted roll 300 and over a top perf-emboss roll 400. The top perf-emboss roll 400 contains perf-emboss projections 405 which contiguously engage with a bottom perf-emboss roll 500. Both rolls contain projections 405 and 505 or teeth in the shape of a polyhedron such as a square, rectangle, diamond or the like. Both rolls are heated to a temperature of from about 150° to about 225° Fahrenheit. Preferably, the top roll 400 is heated to a relatively higher temperature compared with bottom roll 505, i.e., within a range of about 175° to about 225° F.; the bottom roll 505 is relatively cooler, preferably heated to a temperature from about 150° to about 200° F. The teeth or projections 405 and 505 are aligned such that, through the film and absorbent layers, they engage the surfaces of the opposite rolls, i.e., teeth 405 contact surface 510 and teeth 505 contact surface 410. Projections or teeth 405 and 505 should not contact each as rolls 400 and 500 rotate. Rolls 400 and 500 should rotate in opposite directions, as shown by arrows 420 and 520. Preferably, the interference, i.e., the overlap between the teeth should be from about 90 to about 110 mils, with a maximum of about 135 mils apart. Referring to FIG. 7, the perf-embossing step creates a laminate 600 wherein the absorbent layer and the film are fused together.

After the perf-embossing step, a hot-melt applicator 700 applies hot melt adhesive to the absorbent layer side of the absorbent laminate 600 which faces outwardly from the film portion. The adhesive-containing absorbent laminate 600 is run under a weighted roll 800 and breathable barrier film 805 is applied and laminated to the absorbent layer, creating the laminated secondary absorptive layer of this invention.

The combination of the perf-embossed absorbent composite layer, which is three-dimensional in configuration, with the relatively flat barrier film layer results in the creation of “void volume pockets”, i.e., areas of air locate in the spaces created by the embossed surface of the absorbent layer that can provide air circulation, store liquid and provide places where particles of material, such as superabsorbents, may be placed in the secondary layer.

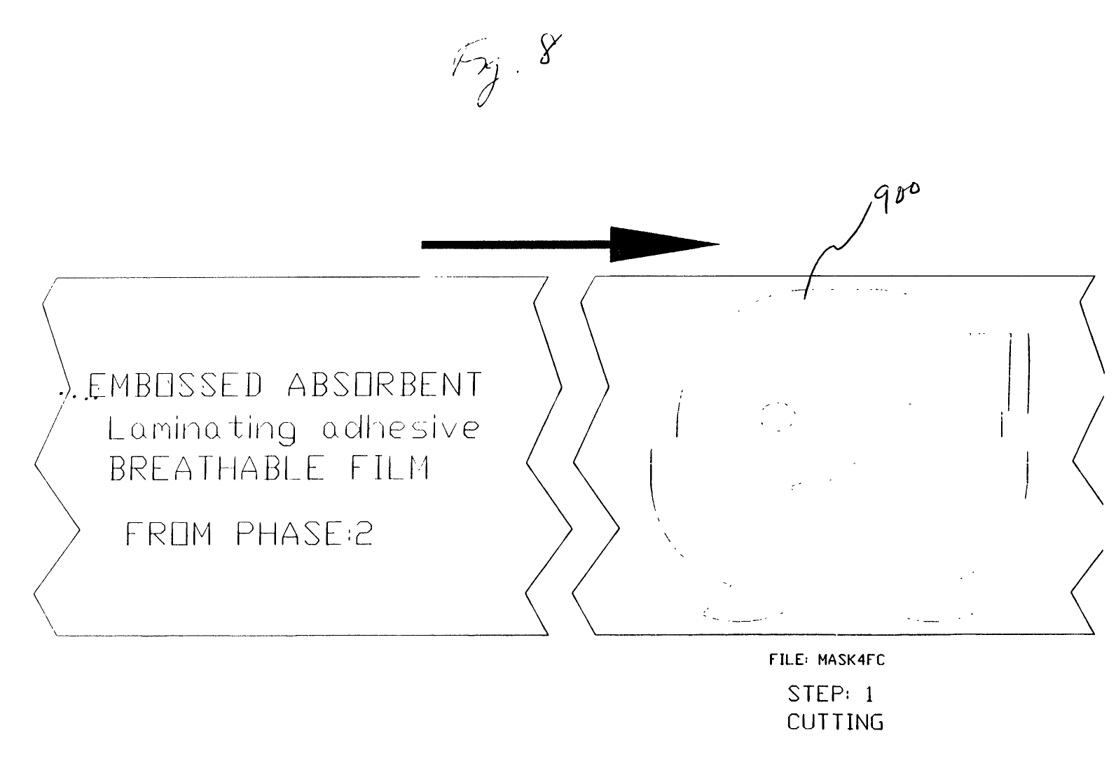

The secondary absorptive laminate layer may be “die-cut” to produce a wide range of personal care and wound or skin care products for applications which require fluid management, e.g., facial masks, panty liners, sanitary napkins, incontinence products, wound dressings and the like. For example, referring to FIG. 8, the absorptive laminate layer may be cut into a face mask 900.

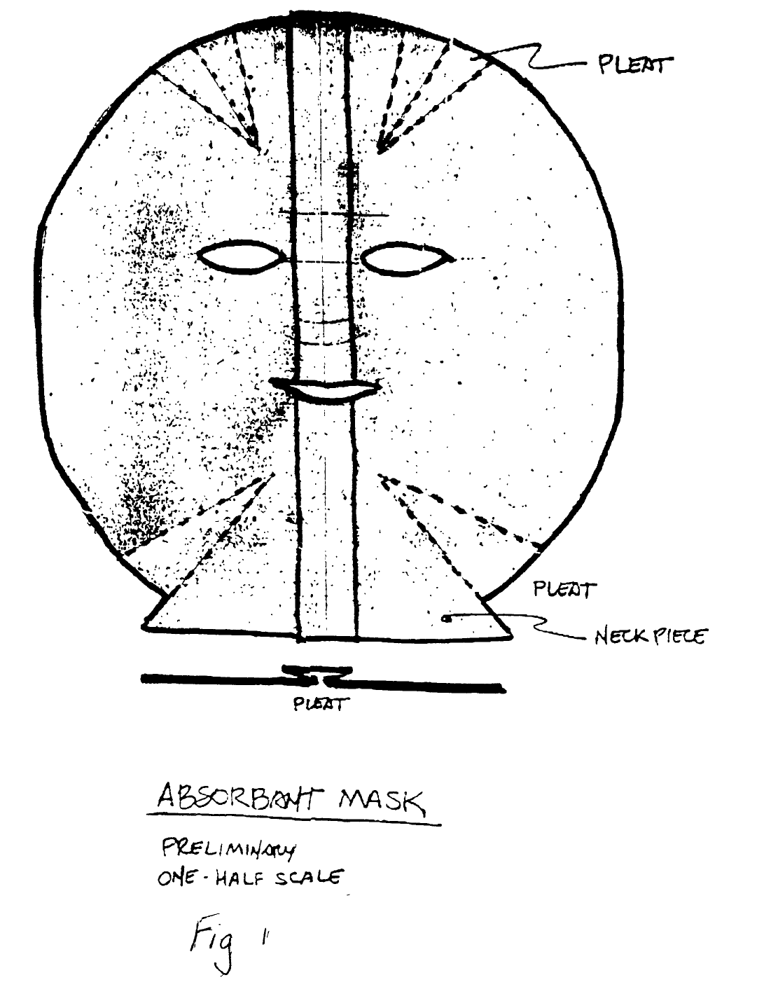

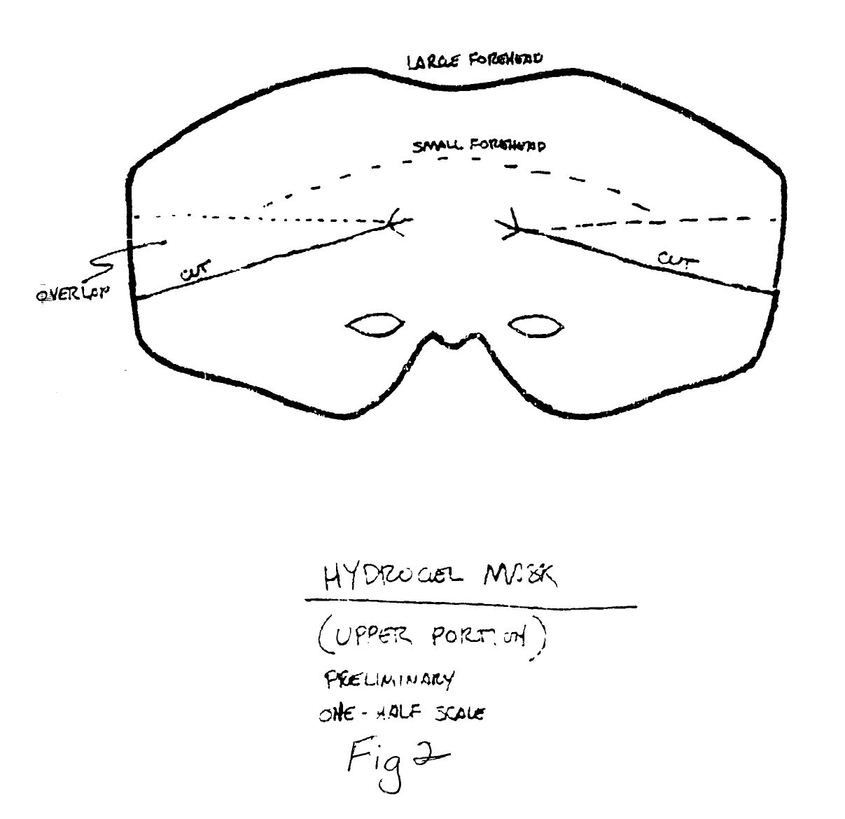

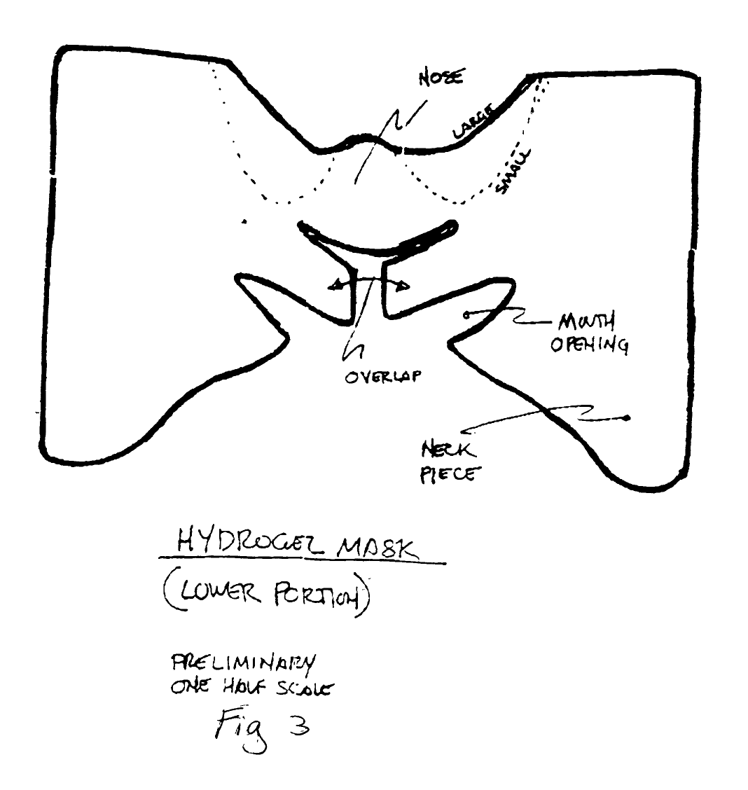

The secondary absorptive layer is preferably formed in the shape of a face with openings for the eyes, nose and mouth. The secondary absorptive layer conforms to facial contours and adheres to the primary absorptive layer, maintaining its position on the face for about 24 to about 36 hours. It may be held in place with retaining straps or bands, as set forth in FIG. 1. FIG. 2 demonstrates a mask according to the process of this invention that permits the formation of a wound dressing from a single flat sheet of secondary absorbent laminate that can be folded into a conformable, three-dimensional product. The openings around the edges, eye, nose and mouth openings may be heat-sealed so as to prevent leakage of fluid and provide comfort for the patient. Score lines 15 provide for folding guides such that the physician or patient can fold and secure the mask around the face in a manner so as to conform it with the shape of the patient's face. It may be secured with a simple band or tape. A nose-piece may be cut that can be placed with tape over the nose-opening to prevent the skin beneath from drying out.

Another method of securement is as follows. This can be accomplished by using three moldable strips that are positioned on the forehead, over the bridge of the nose, and surrounding the lower jaw line. This moldable or bendable material must conform to the contours of the face enabling a dressing or covering to maintain direct contact with the facial surface. The strips should be attached to each other in such a way to ensure that they perform, interdependently, as a unit. This attachment can be a rivet or a hinge allowing movement to duplicate movement of the face and jaw. A flexible, elastic material such as a nonwoven or woven strap is attached to the unit to secure the device to the head. The elastic, flexible strap can attach to the unit at one or more positions ensuring that the unit continues to conform to the contours of the face. The strap may encompass the entire head or be designed in a way to hook behind the ears.

Another embodiment provides a compressible foam supported on a rigid or semi-rigid structure. The support structure could be designed to encompass the perimeter of the face with a bridge extending across the bridge of the nose. The unit can be fixed or hinged to allow for movement of the jaw. The foam should be compressible to hold a dressing or a covering in direct contact with the skin surface. A flexible, elastic material such as a nonwoven or woven strap is attached to the unit to secure the device to the head. The elastic, flexible strap can attach to the unit at one or more positions ensuring that the unit continues to conform to the contours of the face. The strap may encompass the entire head or be designed in a way to hook behind the ears.

After a period of about 24 to 36 hours, the initial wound dressing composed of either the primary and secondary absorptive layers or the secondary layer itself is removed, as the skin will have entered the second phase of returning skin to a functional barrier following resurfacing, the proliferative phase. During this phase, epidermal regeneration is the major goal. Management of the wound during this phase includes providing the optimal temperature and humidity to facilitate healing and minimize patient and discomfort. While early in phase 2, the wound is exuding a small amount of fluid that must be absorbed, by the end of phase 2, the wound healing environment is drier. For the phase of healing, a maintenance layer should be applied to the skin. Preferably, this maintenance layer is composed of a hydrogel mask, which helps to optimize epidermal migration and proliferation.

The hydrogel mask is preferably composed of polyethylene oxide matrix and water crosslinked to form a hydrogel. Its flexibility and form engineering affords flexibility and conformation to the contours of the face, cooling and soothing upon application. It also acts as a “skin substitute”, replacing barrier properties during epidermal regeneration, protecting the neoepidermis prior to maturation. The maintenance layer should be light in weight, and should be capable of cooling and soothing the skin. Preferably, it absorbs up to 3 times its weight in exudate. It should also conform and adhere to facial contours without an adhesive. The maintenance layer should preferably create an optimized environment for epidermal proliferation, migration and maturation, enabling healing to proceed at a rapid rate and should be able to be removed without disturbing regenerated epidermis.

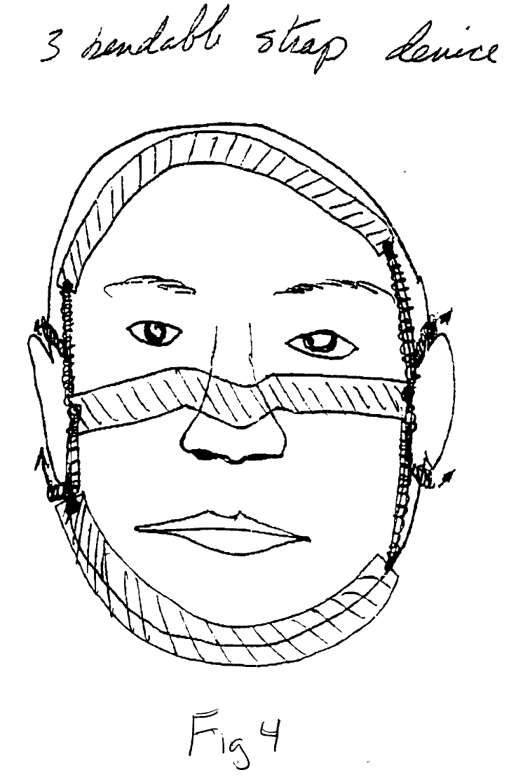

Preferably, the maintenance layer should stay in place with dressing retention strap and may appear as set forth in FIGS. 2 and 3. FIG. 2 depicts an upper facial maintenance layer and FIG. 3 depicts a lower facial maintenance layer. The maintenance layer should be retained on the skin for about 24 to about 36 hours, after which it should be removed.

The final stage of healing following laser resurfacing, deep chemical peeling and dermabrasion involves reestablishing the barrier properties of the skin. Barrier materials should be applied to the skin in this phase of healing in order to assist the skin in reestablishing the skin's barrier properties. Preferably, barrier materials used are composed of Moisturizing compositions. High glycerin moisturizers, for example, have been proven to create a pseudo-barrier, minimizing trans-epidermal water loss and enhancing intercellular lipid fluidity. They have also been shown to stabilize the lipid bilayer, enabling restoration of the stratum corneum barrier function.

More preferably, a concentrated, oil-free emollient containing 40% glycerin to create a pseudo-barrier, protecting the neoepidermis and minimizing trans-epidermal water loss is used. A small amount of such cream is applied twice a day for five days to complete the acute healing program and transition the patient into a maintenance skin care regimen. Most preferably, said barrier material is Norwegian Formula Hand Cream*, fragrance-free formula commercially available from Neutrogena Corporation, Los Angeles, Calif.

Preferably, the barrier material creates a pseudo-barrier to help prevent excessive transepidermal water loss. It maintains increased moisture levels in the skin for up to 17 hours or more to promote comfort and healing, and helps to preserve the lipid bilayer to enable restoration of skin barrier function. The barrier material should create an osmotic environment that is inhospitable to bacterial growth, and normalize desquamation.

Preferably, a kit containing the compositions and apparatus of this invention may be assembled as a kit for use by a physician, containing the following components:

1 set of Fibracol (3 ea), lyophilized collagen dressing
1 ea absorbent cover for Fibracol
1 ea (or potentially two) dressing retention band
1 ea polyethylene oxide (PEO) hydrogel mask
1 ea tube (2.5 oz) Resurface Healing Cream
1 ea tube (2.0 fl oz) Extra Gentle Cleanser
1 ea sample tube (0.25 fl oz) Neutrogena Moisture SPF15
1 ea patient instruction brochure
1 ea physician letter (to patient)
1 set physician package insert

The following examples serve to illustrate, but not to limit, the scope of the apparatus, process and compositions of this invention.

### EXAMPLE 1

A face mask was made in accordance with this invention was made as follows. A polyethylene/ethylene vinyl acetate coextruded film commercially available as XP-11679B from Edison Plastics of Edison, N.J., an absorbent batt containing thermofusible bicomponent fiber having a polyethylene sheath and a polyester or polypropylene core, available as 120.899 from Concert of Thurso, Canada and a breathable polyethylene film available as Br-300 from Clopay of Cincinnati were obtained. The layers of film and batt were perf-embossed as follows. A roll containing the polyethylene/ethylene vinyl acetate film was unwound over a roll containing an absorbent layer, such that the ethylene vinyl acetate side of the film faced the absorbent roll. The two layers were wound around a top perf-emboss roll. The top perf-emboss roll was contiguous with a bottom perf-emboss roll. Both rolls contain projections or teeth in the shape of a hexagon. Both rolls are heated to a temperature of from about 150° to about 225° Fahrenheit. The top roll was heated within a range of about 175° to about 225°; the bottom roll was heated to a temperature from about 150° to about 200°. The distance between the embossments made upon the layer were about 90 to about 110 mils apart. After the perf-embossing step, hot melt adhesive was applied to the outward-facing side of the absorbent core (i.e., the side facing away from the skin-facing film which becomes the acquisition layer in the completed composite) and breathable barrier film was applied and laminated thereto. Photographs of the embossed film with the perf roll magnified 20× in order to view the embossments and perforations better is set forth in FIGS. 5a, 5b and 5c. The slitted openings are apparent in the figures. These slitted openings permit fluid to be absorbed directly into the absorbent core of the laminate without permitting excessive wet-back.

The physical characteristics of the laminate are set forth in Table 1 below.

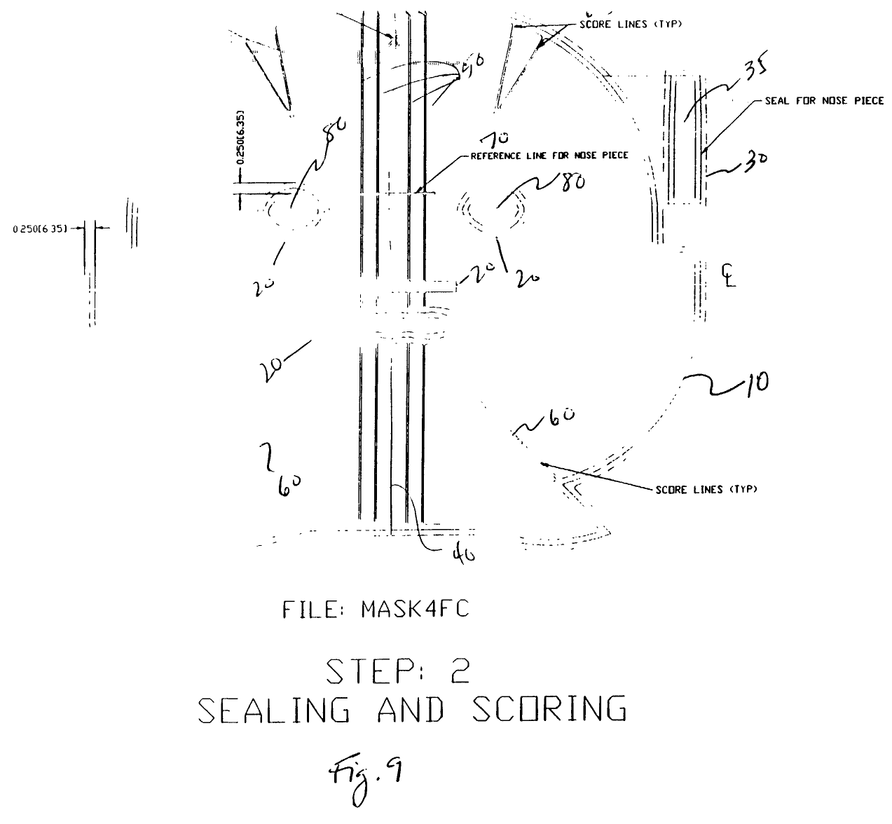

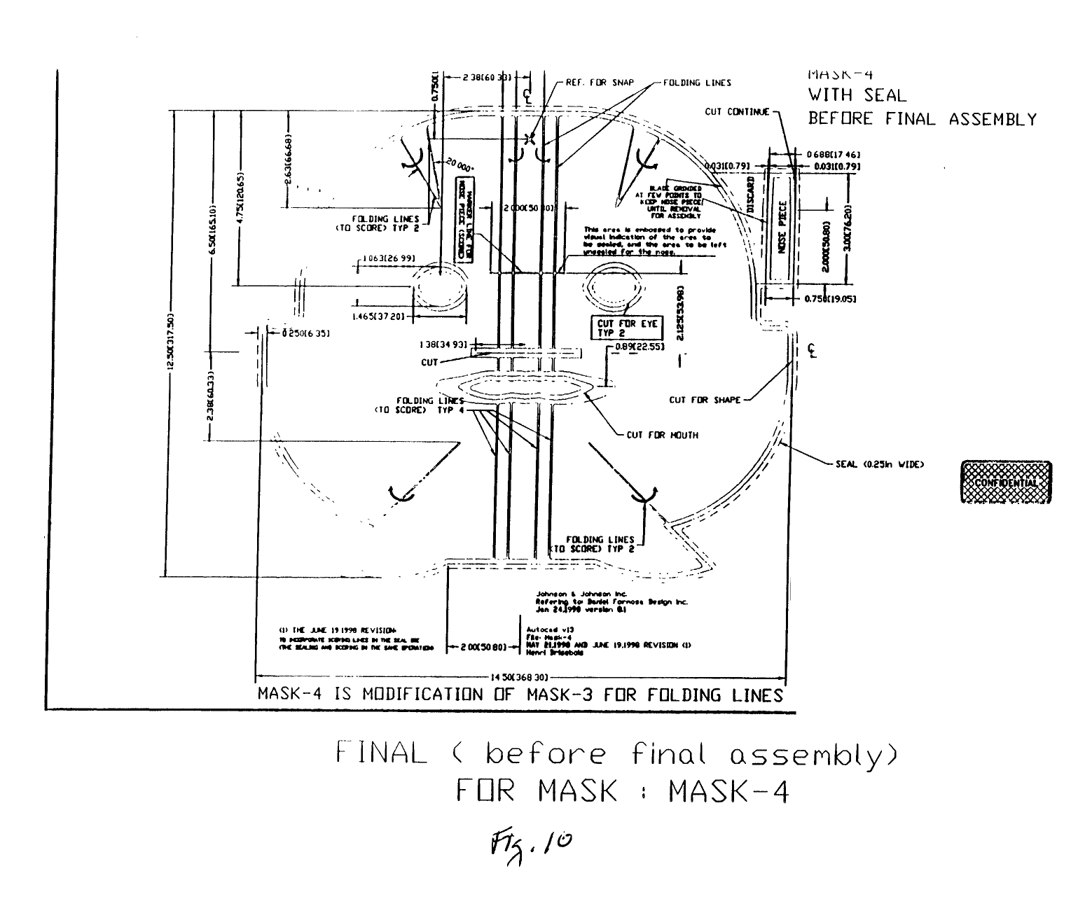

After lamination and perf-embossing, the laminate was cut into a face mask 900 as set forth in FIG. 8. After cutting, the mask blank should be sealed and scored as set forth in FIG. 9. Sealing should be performed around the periphery 10 of the mask as well as at apertures 20 and around the periphery of the nose piece 30. The mask should then be scored at least four straight lines 50 at equidistant points from the perpendicular axis 40 and parallel to perpendicular axis 40. Score lines may actually be seal lines as well; so long as they serve to weaken the laminate for areas for folding to occur more readily than other areas of the batt. Score lines 60 may be made at an acute angle to the perpendicular axis 40. The score lines 50 and 60 permit the mask to be folded easily and conformed to the three-dimensional configuration of the face. Pleats may be made along the score lines 50 by folding the score lines most distant from perpendicular axis 40 either toward the body-facing side of the mask or the outward-facing side of the mask, pivoting the material along the inner-most score line. Likewise, the mask may be folded to make a dart and cause the mask to be in the shape of a three-dimensional configuration along the score lines 60. Score lines 65 also provide the means to create a “dart” or pleat in the mask so as to create a three-dimensional structure. The mask may be accordingly adjusted in size to conform to the size and shape of the patient's head. Folding patterns as set forth above may be found in FIG. 10. The nose piece 35 may be detached and adhesively placed on the outward-facing surface of the mask at reference line 70, along the perpendicular axis 40 and centered between the eye holes 80.

TABLE 1
Face Mask Laminate Physical Characteristics

| Characteristic | Measurement | Test Method | Avg. | STD | Units |
|---|---|---|---|---|---|
| Breathability | MVTR | 1292 | 1273 | 253 | g/m2/day |
| Flexibility | Modified Circular | | | | |
| | Bend | 4032-82 | | | |
| Top | | | 175 | 24 | Peak load- |
| | | | | | 5g |
| Bottom | | | 183 | 23 | Peak load- |
| | | | | | 5g |
| Absorption Rate | Critical Rate | 16140-9 | 80 | 34 | ml/min |
| | (Synthetic Wound Exudate) | | | | |
| Absorption Speed | Penetration Time | W78005 | 20 | 5 | seconds |
| | (Synthetic menstrual fluid) | | | | |
| Absorption Capacity | Saturation Test | | 135 | | g |
| | (Synthetic Wound Exudate) | | | | |

Generally, the flexibility of the composite should be from about 100 to about 250 g as measured by the Modified Circular Bend test, more preferably from about 150 to about 225 g and most preferably from about 170 to about 190 g. The Modified Circular Bend test is the Circular Bend test number ASTM 4032-82 which has been modified as follows. A plunger having a radius of 42 mm, a 2000 g Instron compression cell and a 500 kg reversible load cell are used. Specimens are conditioned by leaving them in a room which is 21±1° C. and 50±2% for a period of 2 hours. The specimens are cut into two specimens of 4 sq. in. in vertical portion. The specimen is placed a the plate of the compression cell and centered in order for the plunger to descend at a rate of 50 cm per minute per full stroke length in the middle of the specimen. The plunger descends, bending a circular portion of the specimen and ascends, leaving the surface of the specimen. The numerical values of the test results are generated by SINTECH TESTWORKS FOR WINDOWS (MTS) software.

The MVTR was measured using test method ASTM E96-90 or E96-E of Clopay of Cincinnati, Ohio.

The composite should have an absorption rate of from about 50 to about 100 ml/min, more preferably from about 75 to about 85 ml/min using synthetic wound exudate having similar viscosity to that of wound exudate (approximately 5-7 centipoise). The composite should have an absorption speed of from about 10 to about 30 seconds using viscous synthetic menstrual fluid having a viscosity of about 30-13.5 centipoise, more preferably from about 15 to about 25 seconds. The absorption capacity should be at least about 100 g, more preferably at least about 120 g.

### EXAMPLE 2

The effectiveness of a wound management regimen in accordance with this invention in returning the skin to normal function following resurfacing procedures was evaluated.

Several variables were measured, including: exudate production, onset of epithelialization, rate of epithelialization, wound cosmesis, ease of use, product performance and effectiveness, presence of microbial flora, presence of unexpected erythema, inflammation, infection, and irritation. Eighteen female subjects, between the ages of 35 and 65 were enrolled in the evaluation.

The study was executed as follows: all wounds were managed similarly with the three-step system of this invention. Immediately following surgery, the subject's treated skin was dressed with FIBRACOL* wound dressing over the entire face. The FIBRACOL* wound dressing was covered with a waterproof, breathable, absorbent cover and held in place with a plastic, anti-fog coated film, supported by open-cell foam, securement device for thirty-six to forty-eight hours (step 1). Following this period, the dressing was removed and the face dressed with NEUTROGENA® RESURFACING gel over the entire surface, covered with a primary and secondary absorbent waterproof, breathable, absorbent cover, and held in place with a plastic, anti-fog coated film, supported by open-cell foam, device for twenty-four to thirty-six hours (step 2). On Day 3 after surgery, the absorbent cover and NEUTROGENA® RESURFACING gel was removed. The face was washed with water and NEUTROGENA® Extra Gentle Cleanser. The Investigator then determined whether the skin is completely epithelialized or another wound dressing is required. If the skin was not epithelialized, the NEUTROGENA® RESURFACING gel (a hydrogel skin dressing available from North American Sterilization and Packaging Company of Franklin, N.J. 07416) was reapplied and covered with another absorbent cover for an additional twenty-four hours. After complete epithelialization, the subject applied a thin layer of the NEUTROGENA® Resurfacing Cream (step 3). The skin cleansing and barrier application procedure was followed six times, evenly spaced throughout the day, for three successive days and nights. Following this regimen, the Investigator determined whether or not the facial skin is completely regenerated. If the wound is not completely healed, the treatment was continued for a period determined by the investigator not to exceed seven days. The subject returned on the last day of the extension period to document complete epithelialization. If the skin was completely healed, the subject began a normal skin cleansing and moisturizing regimen throughout the remainder of the study. The subject returned during the second week and fourth week post surgery for a follow-up visit and a study exit evaluation. The study concluded at the completion of the four-week follow-up visit.

Test Articles included FIBRACOL* collagen-alginate wound dressing, product code 2495, available from Johnson & Johnson MEDICAL, INC. of Arlington, Tex. as the primary dressing. This wound dressing was covered with an waterproof, breathable, absorbent secondary absorbent composite according to this invention for the first 48 hours after surgery. Neutrogena® Resurfacing Gel, a hydrogel skin dressing available from North American Sterilization and Packaging Company of Franklin, N.J. 07416 and covered with a waterproof, breathable, absorbent secondary dressing in accordance with this invention was applied for days 2 and 3 during step 2 of the method. NEUSURFACE HEALING OINTMENT*, manufactured by Enterprises Importfab Inc., Claire, Quebec, Canada, was applied to resurfaced skin exposed to air for days 3 through 6 post-operatively without the absorbent composite cover. RESURFACE HEALING CREAM*, available from Neutrogena Corporation of Los Angeles, Calif. was applied to the resurfaced skin from days 6 through 12 post-operatively.

All subjects underwent a full-face laser resurfacing procedure. All patients included in the study had Ultra-pulse CO₂ or Ultra-pulse CO₂ and Erbium/YAG laser procedures.

Wound dressing assessments were made by the designated study personnel at every visit until complete skin regeneration is observed by the investigator. The dressings were evaluated for the following parameters:

Appearance

5=pristine

4=slightly soiled

3=mildly soiled, limited to 1 or 2 areas

2=moderately soiled, entire surface

1=extensively soiled

Exudate Absorption

5=no exudate present

4=minimal exudate, with extra capacity

3=moderate exudate with complete saturation but without channeling or dripping

2=moderate exudate with slight channeling and dripping

1=excessive exudate with moderate to heavy channeling and dripping

Attachment

4=all sides attached

3=one side detached but wound not exposed

2=two sides detached but wound not exposed

1=two or more sides detached with wound exposure

In-Use Wound Pain

5=no pain

4=minimal pain, not distracting

3=moderate pain, noticeable through day

2=moderate pain causing distraction

1=severe pain necessitating pain medication

After removal of the device by the subject, under the supervision of the appropriate study personnel, the wound repair device was rated on the following parameters:

Ease of Removal

5=effortless

4=minor effort

3=difficult

2=more difficult (removal hampered by product separation)

1=extremely difficult

Pain Associated with Removal

5=no pain

4=minimal pain, not distracting

3=slight distracting pain

2=moderate pain, causing distraction

1=severe pain

Reinjury Upon Removal

5=no reinjury

4=minimal, punctate bleeding

3=minimal, diffuse bleeding

2=moderate, either punctate or diffuse bleeding

1=severe pooling of blood

Epithelialization

5=Wound is completely healed, no dressing necessary

4=Wound is epithelialized and does not require a dressing

3=Wound is greater than 75% epithelialized

2=Wound is greater than 50% epithelialized

1=Wound is greater than 25% epithelialized, appearance of epithelial islands

0=No presence of epithelial islands

A primary statistical analysis was performed of an analysis of variance conducted on the number of days until complete epithelialization to test the hypothesized equivalence of the management system versus control therapy. If equivalence in time to complete epithelialization is rejected, then an appropriate multiple comparison technique will follow to evaluate the relative effectiveness of the various dressings pair-wise. A two-sided significance level will be employed.

Subjective evaluations of dressing functionality and wound appearance were examined descriptively. Performance profiles of means or medians were presented graphically. Evaluations that visually discriminate among the dressings were examined further.

The results of the clinical test are set forth in Tables 1 and 2 below:

TABLE 2.
Skin Regeneration During Trial

| | Percent of Subjects Achieving Score | | | | | |
|---|---|---|---|---|---|---|
| | POD | POD | POD | POD | POD | POD |
| Epithelialization Score | 2 | 3 | 4 | 5 | 6 | 7 |
| >25% Epithelialized | 28 | | | | | |
| >50% Epithelialized | 39 | | | | | |
| >75% Epithelialized | 6 | 6 | | | 100 | 100 |
| No Dressing Required | 0 | 94 | 100 | | 17 | 100 |
| Complete Regeneration | 0 | 0 | 0 | | 83 | 100 |

N = 18

N=18

TABLE 3
Face Mask Performance in Clinical Trial

| | Post-Operative Day 2 | | Post-Operative Day 3 | |
|---|---|---|---|---|
| Characteristic | Score | STD | Score | STD |
| Absorbency | 1.9 | 1 | 3.4 | 1.0 |
| Wound Protection | 3.8 | 1 | 4.3 | 0.9 |
| Protection from Pain | 4.6 | 0.9 | 4.8 | 0.5 |
| During Use | | | | |
| During Removal | 4.6 | 0.6 | 4.8 | 0.4 |
| Fit | 2.7 | 1.2 | 2.7 | 1.0 |
| Wound Appearance | 4.3 | 0.4 | 4.5 | 0.5 |

Scoring: 5 = excellent, 4 = very good, 3 = good, 2 = fair, 1 = poor
N = 20

Scoring: 5=excellent, 4=very good, 3=good, 2=fair, 1=poor

N=20

What is claimed is:

1. A method of promoting the healing of skin comprising the following steps:
(A) applying a primary absorptive skin-facing layer to the skin of a mammal having a wound said primary absorptive skin-facing layer absorbing wound exudate during the early inflammatory phase of wound healing and said primary absorptive skin-facing layer having the characteristics of absorbing fluid from the wound without adhering to the wound and which is sufficiently breathable to permit vapor to escape from the wound or skin site covered;
(B) upon completion of the early inflammatory stage of wound healing, removing said primary absorptive skin-facing layer and applying a maintenance layer which regulates the temperature and humidity of the wound environment during the proliferative phase of wound healing; and
(C) upon the completion of the proliferative phase of wound healing, removing said maintenance layer and applying a barrier material which minimizes trans-epidermal water loss and enhances intercellular lipid fluidity of the skin during the phase in which the barrier properties of the skin are reestablished.

2. A method according to claim 1 wherein step (A) further comprises applying a secondary absorptive layer which is breathable.

3. A method according to claim 1 wherein said primary absorptive layer comprises a composite laminate comprising a skin-facing acquisition facing, an absorbent core associated therewith and a porous, breathable outward-facing layer.

4. A method according to claim 3 wherein said composite laminate comprises a perf-embossed structure.

5. A method according to claim 1 wherein said maintenance layer comprises a hydrogel layer.

6. A method according to claim 1 wherein said barrier material comprises a skin moisturizer composition.

7. A method according to claim 2 wherein said primary absorptive layer comprises a lyophilized collagen-alginate wound dressing.

7. A composite absorbent product comprising:
(a) a skin-facing acquisition facing comprising a film which is capable of being perforated without substantial stretching, said skin-facing acquisition facing having a skin-facing side and an outward-facing side;
(b) an absorbent core contiguously associated with said skin-facing acquisition facing at its outward-facing side, said absorbent core having a skin-facing side and an outward-facing side; and
(c) a barrier film layer contiguously associated with said absorbent core at its the outward-facing side of said absorbent core, said barrier film layer being breathable;
said composite absorbent product having a Peak-load (5g) according to a Modified Circular Bend Procedure of from about 150 to about 250 g and wherein said composite absorbent product contains void volumes which are available for containing fluid.

8. A composite absorbent product according to claim 7 wherein said composite is laminated.

9. A composite absorbent product according to claim 7 wherein said composite is perforated and embossed, creating perforations and embossments in said composite, wherein said perforations are from about 90 to 135 mils apart.

10. A composite absorbent product according to claim 7 wherein said acquisition facing comprises a polyalkylene polymer film, a coextruded polyethylene/ethylene vinyl acetate film or a nonwoven fabric comprising thermofusible fibers having a basis weight of from about 0.5 to about 2 oz/yd².

11. A composite absorbent product according to claim 7 wherein said absorbent core comprises a nonwoven fabric or batt.

12. A composite absorbent product according to claim 11 wherein said nonwoven fabric or batt comprises from about 5 to about a 40% thermofusible bonding fiber and wood pulp fluff.

13. A composite absorbent product according to claim 11 wherein said absorbent core has a basis weight of from about 50 to about 200 g/m².

14. A composite absorbent product according to claim 11 wherein said absorbent core has a density of from about 0.03 to about 0.2 g/cc.

15. A composite absorbent product according to claim 7 wherein said barrier film layer comprises a porous film.

16. A composite absorbent product according to claim 7 wherein said barrier film layer has a Moisture Vapor Transmission Rate of from about 500 to about 7500 g/m²/day.

17. A composite absorbent product according to claim 16 wherein said Moisture Vapor Transmission Rate is from about 100 to about 300 g/m²/day.

18. A composite absorbent product according to claim 15 wherein said barrier film comprises a coextruded polyethylene/ethylene vinyl acetate microporous film.

19. A process for making an absorbent composite laminate comprising the following steps:
(a) providing an acquisition layer and a contiguous absorbent core layer having an acquisition layer-facing side and an outward-facing side which are capable of bonding to each other when heat is applied;
(b) perforating and embossing said acquisition layer and said contiguous absorbent core layer at a temperature of from about 150 to about 225° F., thus fusing said absorbent core layer to said acquisition layer at the acquisition layer-facing side of said absorbent core layer;
(c) applying an adhesive to the outward-facing side of said absorbent core layer and laminating a breathable barrier film to the absorbent core layer.

20. A skin dressing for the face comprising a composite absorbent product comprising:
(a) a skin-facing acquisition facing comprising a film which is capable of being perforated without substantial stretching, said skin-facing acquisition facing having a skin-facing side and an outward-facing side;
(b) an absorbent core contiguously associated with said skin-facing acquisition facing at its outward-facing side, said absorbent core having a skin-facing side and an outward-facing side; and
(c) a barrier film layer contiguously associated with said absorbent core at its the outward-facing side of said absorbent core, said barrier film layer being breathable;
said composite absorbent product having a Peak-load (5 g) according to a Modified Circular Bend Procedure of from about 150 to about 250 g;
said dressing having the approximate shape of a face and containing apertures for eyes, nose and mouth; said dressing further containing score lines for pleats along its perpendicular axis, equidistant from the midline of the dressing and score lines for darts located such that each of said dart score lines is at a 45° angle from said midline, beginning at the peripheral edge of said dressing.

21. A method of making a skin dressing for the face comprising the following steps:
(a) providing an absorptive layer;
(b) cutting a facial-shaped mask in said absorptive layer;
(c) cutting apertures for eyes, nose and mouth in said absorptive layer;
(d) sealing the peripheral edges of said mask and said apertures using radiant energy or adhesive;
(e) making at least two score lines on a vertical axis parallel with the midline of said mask, said score lines being equidistant from said midline;
(f) making at least four score lines at 45° angles from said midline, at least one in each quadrant of said mask; and
(g) folding said mask at said score lines to form at least one vertical pleat and at least four darts.
